(19) United States
(12) Patent Application Publication
(10) Pub. No.: US 2009/0136423 A1
(43) Pub. Date: May 28, 2009

(54) Methods for assessing psychotic disorders

(75) Inventors: Barbara Sahakian, Cambridge (GB); Edward Bullmore, Cambridge (GB); Jennifer Barnett, Cambridge (GB); Peter Jones, Cambridge (GB)

Correspondence Address: WOLF GREENFIELD & SACKS, P.C., 600 ATLANTIC AVENUE, BOSTON, MA, 02210-2206, US

(73) Assignee: Cambridge Enterprise Limited

(21) Appl. No.: 11/990,825

(22) PCT Filed: Aug. 23, 2005
(86) PCT No.: PCT/GB2005/003279

§ 371 Date: Feb. 22, 2008

(51) Int. Cl. A61K 49/00; A61B 5/16

(52) U.S. Cl. 424/9.1; 600/558

(57) ABSTRACT

This invention relates to the assessment of individuals with first-episode psychosis (FEP) or an at risk mental state (ARMS) using visuospatial associative learning tests such as paired associates learning (PAL). Visuospatial associative learning ability as determined by such tests may be prognostic of the severity of psychosis in FEP patients and diagnostic of psychosis in ARMS patients.

This invention relates to methods and means for assessing individuals with first-episode psychosis.

Two of the most striking cognitive impairments in psychosis are in memory and executive functions. Although there have been suggestions of extreme memory impairment in schizophrenia (e.g. McKenna et al 1990), much recent research has focused on executive deficits. One reason executive deficits in schizophrenia have captured research interest is their potential as trait markers of genetic risk for psychosis. Impaired executive function is found in the healthy relatives of people with schizophrenia (Byrne et al 1999; Cannon et al 1994; Egan et al 2001; Kremen et al 1994; Wolf et al 2002). However, in psychotic disorders executive deficits may reflect disease progression. The IDED attention-shifting task from the Cambridge Neuropsychological Test Automated Battery (CANTAB) is a measure of executive functioning; the ‘extra-dimensional’ shift (EDS) stage of the task is conceptually akin to the WCST. Performance at the EDS is worse in chronic schizophrenia than in first-episode patients (Elliott et al 1995; Hutton et al 1998). Furthermore, performance has been associated with duration of untreated psychosis, suggesting a process of pathological cognitive decline (Joyce et al 2002).

Suggested cognitive markers of decline in psychosis are not limited to executive function. Wood et al (2002) found significant impairment on the CANTAB visuospatial paired associative learning (PAL) task in established schizophrenia, with less impairment in first-episode psychosis, however outcome or prognosis was not measured in this study. One explanation may be a progressive decline in associative learning ability throughout the disorder; this is supported by neuroimaging evidence of structural change in the medial temporal lobe region during the transition from at-risk mental state to psychosis (Pantelis et al 2003a).

The present inventors have recognised that performance in visuospatial associative learning tests by patients with first episode psychosis is associated with prognosis and is independent both of executive function, for example as determined by IDED attention-shifting, and the duration of the psychosis prior to treatment.

One aspect of the invention provides a method for the assessment of an individual having a first episode psychosis comprising;
determining the visuospatial associative learning ability of said individual,
wherein said ability is indicative of the prognosis of the first episode psychosis in the individual.

For example, low visuospatial associative learning ability relative to controls may be indicative of a negative prognosis for the individual, whereas normal or high visuospatial associative learning ability may be indicative of a positive prognosis.

A negative prognosis is a prognosis associated with a poor clinical outcome (such as, for example, severe symptoms, long duration and increased risk of recurrence of the psychosis). Conversely, a positive prognosis is associated with a good clinical outcome (such as, for example, mild symptoms, short duration and reduced risk of recurrence).

Methods of the invention may be useful in determining the severity of the psychosis and/or the prognosis in the individual.

Visuospatial associative learning ability is preferably assessed using a single test, for example a paired associates learning test, preferably a non-verbal paired associates learning test. Various forms of paired associates learning test are known in the art. In preferred embodiments, the Cambridge Neuropsychological Test Automated Battery (CANTAB: Cambridge Cognition Ltd, Cambridge UK) visuospatial paired associates learning (PAL) test may be used (Sahakian et al. (1988) Brain 111: 695-718).

CANTAB PAL is a precisely defined cognitive test which is well-known in the art. It involves the sequential display of 1, 2, 3, 6 or 8 patterns in boxes on a display. Each pattern is then presented in the centre of the display and the subject is required to touch the box in which the pattern was previously seen. If all the responses are correct, the test moves on to the next stage; an incorrect response results in all the patterns being redisplayed in their original locations, followed by another recall phase. The task terminates after 10 presentations and recall phases if all patterns have not been placed correctly. The test may be scored in a variety of ways to produce a visuospatial associative learning score, including for example number of stages passed. Preferably, in methods of the invention, the test is scored by the total number of errors made.

Visuospatial associative learning ability may be expressed as a visuospatial associative learning score, which is determined from the responses of the individual to the visuospatial associative learning test. For example, the score may represent the total number of errors. Alternate methods may include the number of errors at the 6 and/or 8 pattern stage of a PAL test. The visuospatial associative learning score may be adjusted for the age and IQ of the individual. A method may include assessing the IQ of the individual.

The IQ of the individual may be assessed using conventional testing methods, including for example NART (Nelson H (1982). The National Adult Reading Test. Windsor, UK: NFER Nelson)

The results herein show that visuospatial associative learning ability in first episode psychosis is not related to the duration of the untreated psychosis. Preferably, the score is not adjusted for the duration of untreated psychosis.

A low visuospatial associative learning score relative to controls is indicative of severe psychosis and/or a negative prognosis.

For example, the visuospatial associative learning score may be compared to a predetermined threshold value. A score which is less than the predetermined threshold value is considered low and indicative of severe psychotic disorder and/or a negative prognosis. In some embodiments, the predetermined threshold value may be adjusted for the age and IQ of the individual. For example, in the CANTAB PAL test, the threshold value may be fixed as a z-score below −2 according to manufacturer's normative data, which automatically adjusts for each subject's age and NART score.

In some embodiments, the severity of the psychotic disorder or its prognosis may be determined from the test scores, age and IQ of the individual, using a predictive model.

A suitable predictive model may be produced from the visuospatial associative learning ability scores of a sample of early stage psychosis individuals who are subsequently monitored over time for the state of the psychotic disorder.

A first episode psychosis identified as severe using the present methods may display increased symptom levels and reduced global function, relative to psychoses which are not identified as severe.

A method of producing a predictive psychosis algorithm or model may comprise;
assessing the visuospatial associative learning ability of a sample of individuals having a first episode psychosis, to produce visuospatial associative learning scores for each member of said sample;
monitoring the progress of the psychosis in each of said members over a time course to determine the clinical outcome for each of said members, and;
relating scores, age and IQ of each of said individuals with the clinical outcomes of the psychosis in said individuals to produce a predictive algorithm which relates said test scores, age and IQ to said clinical outcomes.

Clinical outcome may be assessed using standard techniques, including the Global Assessment of Function (American Psychiatric Association 1994) and the Structured Clinical Interview for DSM-IV (First et al 1997), and clinical rating scales, such as the Beck Depression Inventory (BDI, Beck and Steer 1987), Young Mania Scale (Young et al 1978) and the Positive and Negative Syndrome Scale (PANSS, Kay et al 1987).

An individual having a first episode psychosis may then be assessed by producing a visuospatial associative learning score for the individual as described above; and,
applying the predictive algorithm to the score and the IQ and age of the individual to determine the prognosis for the individual (i.e. the probable clinical outcome).

The severity or prognosis may be expressed as a grade. Preferably, the grade corresponds to clinical scales which are well known in the art.

A method may further comprise identifying the individual as having a severe form of psychosis or identifying the individual as having a psychosis with a negative prognosis.

For example, the individual may be identified from the grade assigned to the individual. An individual with a grade indicative of a negative prognosis may be identified as having a psychosis with a negative prognosis.

An individual identified as having a severe psychosis and/or a psychosis with a negative prognosis using a method of the invention may be targeted or prioritised for cognitive enhancement, psychological, rehabilitative, or other therapeutic treatment.

An individual having a first episode psychosis may have an unimpaired executive function, or an impaired executive function, for example as defined by an IDED attention-shifting test (e.g. CANTAB IDED)

A method described herein may comprise the initial step of identifying an individual as having a first episode psychosis.

An individual having a first-episode psychosis may be identified by a medical practitioner using standard neuropsychiatric diagnostic criteria for first-episode psychosis (FEP). For example, the individual may display one or more symptoms or behaviours characteristic of psychosis. In some embodiments, individuals suitable for assessment in accordance with the present methods may include individuals identified as having an at-risk mental state (ARMS) using standard neuropsychiatric diagnostic criteria.

Suitable neuropsychiatric diagnostic criteria are set out, for example in the Diagnostic and Statistical Manual of Mental Disorders (text revision), American Psychiatric Association (2000) American Psychiatric Publishing Inc (DSM-IV-TR).

The first episode psychosis may have a more specific diagnosis, for example schizophrenia, schizoaffective disorder, delusional disorder, bipolar disorder, psychotic depression or brief psychotic disorder. In some embodiments, the first episode psychosis may be a psychosis other than schizophrenia. Alternatively, the first episode psychosis may not have a more specific diagnosis.

Individuals identified by the present methods as having a severe form of psychosis or a negative prognosis may be assessed further using other neuropsychological and diagnostic criteria. The individual may, for example, be subjected to increased monitoring and/or assessed for anti-psychotic, cognitive enhancing or other (e.g. psychological) treatment.

The methods described herein may also be useful in the development of suitable treatments for individuals with early stage psychotic disorders. A method of identifying a therapeutic agent useful in the treatment of an early stage psychotic disorder may comprise;
administering a test compound to an individual having a first episode psychosis, and;
determining the visuospatial associative learning ability of said individual,
wherein an increase or improvement in visuospatial associative learning ability following said administration is indicative that the agent is effective in the treatment of the cognitive deficit in psychosis.

Early stage psychosis individuals and methods of determining the visuospatial associative learning ability are described elsewhere herein.

Visuospatial associative learning may be tested at two or more time points. Changes in visuospatial associative learning ability between the time points may be determined.

The treatment may be monitored periodically, for example weekly or monthly to assess its effect. Visuospatial associative learning may thus be assessed at a number of time points during the treatment. Visuospatial associative learning ability may also be determined before and after the treatment.

Further aspects of the invention provide: (i) computer-readable code for performing a method for the prognosis of a psychotic disorder in an individual with a first episode psychosis as described herein, (ii) a computer program product carrying such computer-readable code, and (iii) a computer system configured to perform a method for the prognosis of a psychosis in an individual with a first episode psychosis as described herein.

The term “computer program product” includes any computer readable medium or media which can be read and accessed directly by a computer. Typical media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

A typical computer system of the present invention comprises a central processing unit (CPU), input means, output means and data storage means (such as RAM). A monitor or other image display is preferably provided.

The input means preferably comprises a touch sensitive monitor or other graphic interface device that allows the selection of displayed graphics or elements by the subject, for example in a paired associates learning test, in particular a non-verbal paired associates learning test, such as CANTAB PAL.

For example, a computer system may comprise a processor adapted to perform a method of the invention. For example the processor may be adapted to:
i. determine the visuospatial associative learning ability of an individual having a first episode psychosis,
ii. produce a visuospatial associative learning score for the individual, and,
iii. correlate said score with the severity and/or prognosis of psychosis in said individual.

The visuospatial associative learning ability of the individual may be determined by performing a paired associates learning test, in particular a non-verbal paired associates learning test, such as CANTAB PAL, and recording the responses entered by the individual via the input means. Suitable input means include a keyboard or more preferably a touch sensitive monitor.

The severity and/or prognosis of the psychosis may be expressed as a grade, which is produced by the processor from the visuospatial associative learning score of the individual, for example using a predictive algorithm or by comparison with a threshold value stored in the processor or data storage means.

The threshold value in a CANTAB PAL test may, for example, be fixed as a z-score below −2 according to manufacturer's normative data. A predictive algorithm may be generated from the visuospatial associative learning scores of a population of individuals, for example in a database, as described above.

The grade may for example, correspond to known clinical scales and may be indicative of the severity, prognosis and/or likely clinical outcome of the psychosis.

The visuospatial associative learning score and/or the threshold values may be adjusted for age and/or IQ to produce the severity and/or prognosis grade. The processor may provide for the entry of the age and/or IQ of the individual via the input means.

In some embodiments, the processor may be adapted to determine the IQ of the individual, for example by performing an IQ test and recording the responses of the individual. Suitable IQ tests are known in the art.

The processor may further be adapted to adjust the threshold value or the visuospatial associative learning score for the individual's age and predicted IQ.

The severity and/or prognosis grade may be stored in the processor or data storage means and/or displayed by the computer system.

The processor may be adapted to test the visuospatial associative learning ability of the individual by means of a paired associates learning test, such as the CANTAB PAL test. Computer-implemented PAL tests are well known in the art.

The data storage means may comprise a memory device for storing the visuospatial associative learning scores and/or calculated severity and/or prognosis grades from the individual. The memory device may be adapted for storing scores and calculated grades from a number of different individuals. Statistics and data derived from these scores and grades may be stored on another or the same memory device, and/or may be sent to an output device or displayed on a monitor.

Another aspect of the invention provides a test device for assessing an individual having a first episode psychosis comprising a display, a graphic interface and a processor adapted for use in a method described herein.

The present data shows that deficits in visuospatial associative learning are characteristic of early stage psychosis. The visuospatial associative learning performance of an individual having an ‘at risk mental state’ (ARMS) may therefore be useful as a diagnostic tool to indicate whether or not the individual is suffering from psychosis.

An aspect of the invention provides a method for determining the presence of psychosis in an individual having an at risk mental state (ARMS) comprising;
determining the visuospatial associative learning ability of said individual,
wherein the visuospatial associative learning ability is indicative of the presence or absence of psychosis.

A reduction in visuospatial associative learning relative to controls may be indicative of the presence of psychosis.

Visuospatial associative learning ability may be determined as described above to produce a visuospatial associative learning score.

An individual having an at risk mental state (ARMS) may be identified by a medical practitioner using standard neuropsychiatric diagnostic criteria for ARMS. For example, the individual may display one or more symptoms or behaviours ARMS. Suitable neuropsychiatric diagnostic criteria are set out, for example in the Diagnostic and Statistical Manual of Mental Disorders (text revision), American Psychiatric Association (2000) American Psychiatric Publishing Inc (DSM-IV-TR).

The presence or absence of psychosis in the individual may be determined from the visuospatial associative learning score. For example, a low visuospatial associative learning score relative to controls may be indicative of the presence of psychosis.

A method may comprise identifying the individual as having a psychosis.

The individual identified by a method described herein as having a psychosis may be monitored and/or assessed further using other neuropsychological and diagnostic criteria. For example, the individual may be further identified as having a specific psychotic condition, for example a condition selected from the group consisting of schizophrenia, schizoaffective disorder, delusional disorder, bipolar disorder, psychotic depression and brief psychotic disorder.

Following, identification of psychosis, the individual may be treated using anti-psychotic, cognitive enhancing or other (e.g. psychological) therapies. Suitable therapies are well known in the art.

Other aspects of the invention relate to a computer system comprising a display and a processor adapted for use in a method for determining the presence of psychosis in an individual as described above, a computer program product carrying computer-readable code for performing such a method, and a computer system configured to perform the such a method.

A test device for assessing or diagnosing an individual for psychosis in accordance with the invention may comprise a display, a graphic interface and a processor adapted as described above.

Computer systems, displays, graphic interfaces, processors and computer program products are all described in more detail above.

Various further aspects and embodiments of the present invention will be apparent to those skilled in the art in view of the present disclosure. All documents mentioned in this specification are incorporated herein by reference in their entirety.

The invention encompasses each and every combination and sub-combination of the features that are described above.

Certain aspects and embodiments of the invention will now be illustrated by way of example and with reference to the figures described above and tables described below.

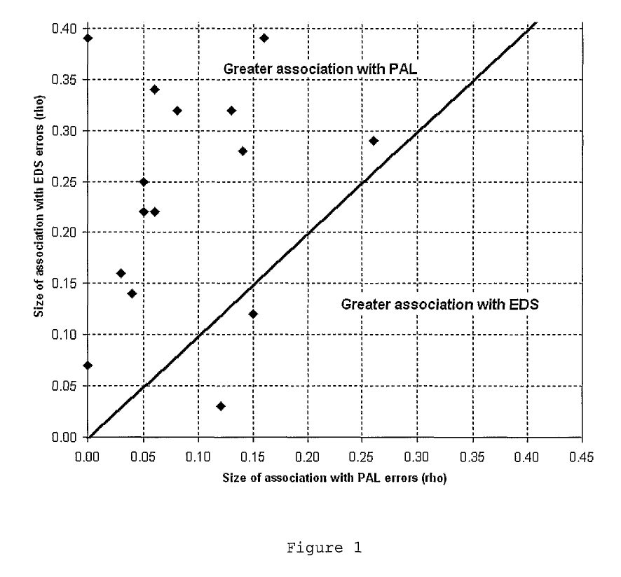

FIG. 1 shows the magnitude of associations between Pal and EDS errors, and clinical, functional and demographic measures shown in Table 1.

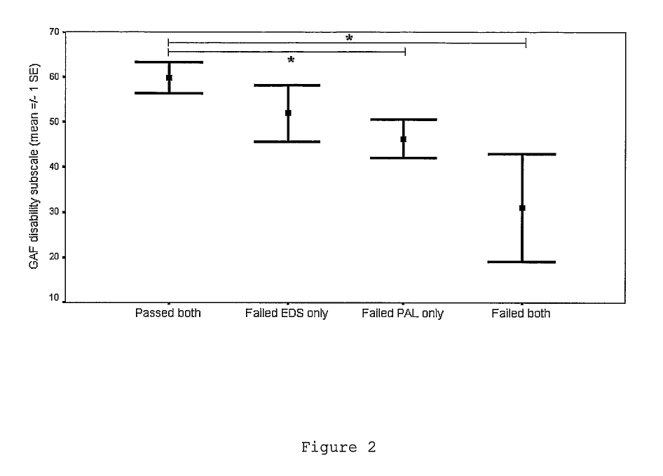

FIG. 2 shows the GAF disability subscale scores of cognitively-dissociated patient groups (mean+/−1 SEM). *Group differences significant at p<0.05.

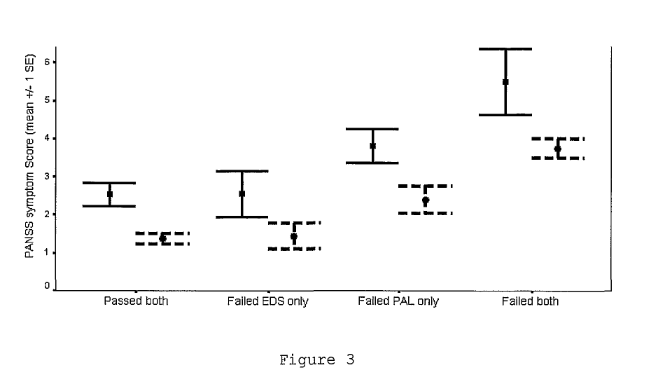

FIG. 3 shows PANSS insight scores (bold lines) and stereotyped thinking scores (broken lines) of cognitively-dissociated patient groups (mean+/−1 SEM). Significant differences (p<0.05) in both cases between ‘Passed both’ and ‘Failed PAL only’, ‘Passed both’ and ‘Failed both’, and ‘Failed EDS only and Failed both’.

Table 1 shows Spearman's correlations between PAL and IDED error scores, and clinical and functional measures.

Table 2 shows Between-group comparisons (t-tests/Mann-Whitney) of demographic, clinical and functional comparisons between patients who pass (n=34) versus fail (n=27) the PAL test.

### Methods

### Design

CAMEO (www.cameo.nhs.uk) is a specialist NHS service for people in Cambridge and South Cambridgeshire who are experiencing first-episode psychosis (FEP) or who are thought to be in an ‘at-risk mental state’ (ARMS) for psychosis. On referral to CAMEO, patients receive a comprehensive clinical assessment including a one-hour neuropsychological battery.

### Participants

Patients referred to the CAMEO service are categorised as FEP or ARMS according to the following criteria:

### First-Episode Psychosis (FEP):

Patients with psychotic symptoms or negative symptoms for the first time; patients with psychotic symptoms with previous untreated episodes, or who have been treated for less than 6 weeks with anti-psychotic medication. Suspected drug-induced psychosis, affective psychoses and dual diagnoses are included.

### At-Risk Mental State (ARMS):

Attenuated psychotic symptoms (odd beliefs, magical thinking, ideas of reference, paranoid ideation, perceptual disturbances) occurring several times a week; transient psychotic symptoms; and ‘trait plus state’ cases who show a family history or vulnerability such as head injury, coupled with a recent significant change in mental state.

Seventy-five consecutive referrals were assessed by the CAMEO service. Most were assessed immediately upon referral to CAMEO, however some (n=18) had been referred prior to the start of neuropsychological assessment and so were seen later than their initial referral. Most patients had been receiving antipsychotic (generally atypical) medication for a short period before assessment. A small minority of patients were not receiving antipsychotic medication, however a substantial proportion was receiving mood-stabilisers or anti-depressants. Local research ethics committee approval was gained to use the clinical data collected in CAMEO for research purposes.

### Neuropsychological Measures

The National Adult Reading Test (NART, Nelson 1982) was used to estimate premorbid IQ. Neuropsychological tasks were taken from the CANTAB, a battery of neuropsychological paradigms presented on a touch-sensitive screen (Sahakian and Owen 1992). The two tasks were presented as part of a longer battery, which took around one hour in total. IDED was presented first in each case.

### IDED Attention Shifting Task (Executive Measure)

The IDED task (Roberts et al 1988) is an attentional set-shifting paradigm. During the conceptually-crucial extra-dimensional shift stage (EDS), divergent thinking is required in order to shift attention away from a previously-correct stimulus dimension to a novel (previously irrelevant) one. The number of errors at the EDS stage was used as the measure of executive functioning.

### Visuospatial Paired Associative Learning Task and Memory Task (PAL)

The CANTAB PAL task, (Sahakian et al 1988) requires subjects to associate visual patterns that cannot easily be verbalized with spatial locations on the computer screen. There are four levels of difficulty; the number of errors at each level is recorded. For participants who fail to complete all levels, an adjusted total is calculated that allows for errors predicted in the stages that were not attempted.

For both tests, an arbitrary “failure” point was fixed as a z-score below −2 according to manufacturer's normative data, which automatically adjusts for each subject's age and NART score. This arbitrary level was chosen to reflect a criterion of very poor performance relative to the norm, and to avoid the difficulties of cut-offs based on the distribution of errors found in the patient sample.

### Clinical and Functional Measures

Clinical rating scales included the Beck Depression Inventory (BDI, Beck and Steer 1987), Young Mania Scale (Young et al 1978) and the Positive and Negative Syndrome Scale (PANSS, Kay et al 1987). The following clinician-rated symptoms included in the PANSS scales were considered separately as well as in the composite scores: N5 (abstract thinking), N6 (flow of conversation), N7 (stereotyped thinking), G11 (poor attention), G12 (insight). The Global Assessment of Function (American Psychiatric Association 1994) was also used, and patients were accorded a clinical diagnosis using the Structured Clinical Interview for DSM-IV (First et al 1997). All clinician-ratings were completed blind to neuropsychological test performance.

Duration of untreated psychosis was assessed from patient and carer report and medical records where available. Two estimates were taken; the first reflected the time that current psychotic symptoms had been present without remittance before anti-psychotic drug treatment was started i.e. duration of untreated psychosis for this episode only. The second was the time between the first psychotic symptom ever experienced and CAMEO cognitive assessment. Where reports from different sources conflicted, the longer duration was taken. These two measures were chosen to provide an estimate of both the long-term history of psychotic symptoms, and the short-term duration of full psychosis, both of which might affect cognitive performance.

### Analyses

To estimate the extent to which performance on the PAL and IDED tests was dissociated, a probabilistic approach was used. We estimated the probability of failing both tests given the probability of failing each test independently. This avoids issues of test sensitivity since the overall rates of failure on each task are irrelevant.

To examine whether there were clinical and functional differences between patients based on their cognitive status, several approaches were employed. First, Spearman's correlations were calculated between total error scores for PAL and the IDED EDS stage, and the clinical and functional measures. Second, the patients were divided into groups based on their status as failing both tests, failing neither test, only failing PAL or only failing IDED. Due to inequality in group sizes, these groups were then compared using non-parametric Kruskal-Wallis tests for multiple-group comparisons, and Mann-Whitney U tests for comparisons between two groups. These comparisons lacked statistical power due to the small sample sizes in some groups. Consequently, comparisons were then made on the basis of PAL or IDED failure regardless of status on the other test. Comparisons between PAL-passers versus PAL-failers were made with t-tests (negative symptoms were first normalized using an inverse square root transform); comparisons between PANSS ‘cognitive symptoms’ and all comparisons between IDED pass/fail groups were made using non-parametric Mann-Whitney tests.

### Results

### Patient Diagnoses and Demographic Effects.

The group comprised 11 ARMS cases and 64 FEP, of whom 8 ARMS and 11 FEP cases were women. The mean age for the group was 26.8 years (SD 9.54) and mean NART-predicted IQ was 109.7 (SD 8.36). Four FEP patients did not complete both IDED and PAL tasks at cognitive assessment due to time constraints or the patient being too unwell, leaving 71 patients included in subsequent analyses.

CAMEO patients often have too short a history of psychosis to be fully diagnosed at first assessment. Nonetheless, SCID diagnoses broke down as follows: schizophrenia (n=13), schizoaffective disorder (2) delusional disorder (3), bipolar disorders (11), major depressive episode (12), psychosis not otherwise specified or brief psychotic disorder (23), psychotic symptoms present but not diagnostically significant (6). One patient was too unwell to complete the SCID.

Median duration of untreated psychosis for this episode was 7.5 months with a range from 0-288 months. Mean length of time between first ever psychotic symptom and cognitive testing was 2.0 years, with a range from 0-27 years.

There were no differences between males and females on the number of EDS errors (z=−0.398, p>0.05) or PAL errors (z=−1.183, p>0.05) made. No differences were found between the number of EDS errors (χ²=4.663, DF 5, p>0.05) or PAL errors (χ²=3.154, DF 5, p>0.05) by SCID diagnosis. Spearman's correlations between age and NART-predicted IQ and PAL and EDS errors found no significant associations.

### Performance on IDED and PAL Tasks

On the IDED task, all patients successfully completed all stages prior to the EDS. Thirteen patients failed to complete the EDS while eight patients completed the EDS but did not then complete the final stage. Errors at both EDS and pre-EDS stages were not normally distributed; the median of EDS errors was 6 (mode=3, range 0-32 errors), while median errors prior to the EDS stage was 7 (mode=4, range 3-21). EDS and pre-EDS errors were not associated (Spearman's rho=−0.08). Fifty-one patients completed the PAL task, with 14 completing the six- but not eight-box stage, and a small minority (n=6) dropping out before completing the six-box task. Total error scores were adjusted for patients who failed to complete all the stages; the median of adjusted errors was 15 (mode=18, range 0-155).

### Dissociation Between PAL and IDED Tests

Of the 71 patients who completed both tasks, 23 (32%) failed PAL yet passed IDED, while 9 (13%) showed the opposite pattern. Four patients (6%) failed both tasks while the remaining 35 (49%) passed both tests.

The degree of dissociation between PAL and IDED test failure was estimated probabilistically as described above. If performance on the tasks were dissociated, the probability of failing both tests would be 6.95%, given the prevalence of failure on each test. The number of patients in the sample who did fail both tests was, in fact, 5.63%, implying no association between tests, i.e., a patient who failed one test was not more likely to fail the other. In addition, Spearman's correlations between PAL and EDS errors showed no significant association (r=0.143, p>0.05).

### Clinical and Functional Associations

Associations between the cognitive test scores and clinical and functional measures can be seen in Table 1 and are summarised in FIG. 1. PAL errors correlated with a number of clinical and functional measures including PANSS Negative symptom scores and GAF disability. EDS errors did not correlate strongly with any clinical measures, however there was a modest correlation with abstract thinking.

### Dissociating the Effect of PAL Failure Versus IDED Failure

Significant differences were found in GAF disability (χ²=10.50, DF 3, p<0.05), stereotyped thinking (χ²=15.03, DF 3, p<0.01) and insight (χ²=9.57, DF 3, p<0.05) between groups defined by their performance on both tests (“pass both”, “fail both”, “fail PAL only”, “fail IDED only”) tests. Post-hoc Mann-Whitney U tests revealed that the significant differences were between “fail PAL only” and “pass both” groups, between “fail EDS only” and “fail both”, and between “fail both” and “pass both” groups (see FIGS. 2 and 3). In all cases the group that failed PAL performed non-significantly better than the group that failed both tests but worse than the group that failed the EDS only, who could not be differentiated from the group that passed both tests.

Direct comparison of patients that were doubly dissociated in terms of their PAL and IDED performance was statistically underpowered due to unequal group sizes. Kruskal-Wallis tests revealed no differences between these two groups in terms of their clinical or function scores.

### Effect of PAL-Failure Alone

Comparisons between PAL-passers and PAL-failers were made using t-tests (see Table 2). Significant differences were found in some symptom rating scores, with PAL-failers showing higher PANSS negative and general psychopathology scores, and trends towards higher positive symptom and mania scores. Some of the differences in PANSS scores may reflect differences in “cognitive” symptom levels, as PAL-passers had lower symptom scores on the insight, stereotyped thinking and attention ratings. PAL-passers showed better functioning on the GAF total and disability ratings.

### Effect of IDED-Failure Alone

No significant clinical or functional differences were found between those who passed versus failed the IDED EDS test.

### Duration of Illness

There were no differences between groups defined by PAL and EDS performance in either the duration of untreated psychosis for this episode (χ²=3.152, DF 3, p>0.05) or in the length of time between first psychotic symptoms appearing and cognitive testing (χ²−1.705, DF 3, p>0.05). This was not simply due to low power: Spearman's correlations between PAL and EDS errors and the two measures of illness duration were all less than 0.2.

In early psychosis patients, failure on the memory and executive tasks was found to be dissociated, in that patients were no more likely to fail both tasks than would be expected by the overall failure rates on the individual tests. This finding is not affected by differences in failure rates on the two tasks.

Patients who failed the PAL task were clinically worse-off than those who passed, while this distinction was not found using the IDED task. Failure on the PAL test was associated with higher PANSS general and negative symptoms and lower GAF score. In addition, there were trends towards increased mania and positive symptoms. This provides indication that PAL failure detects patients with a globally more severe clinical presentation. Poor associative learning and memory may be a cognitive marker of illness severity, while EDS failure, which is not associated with clinical presentation, may reflect a more long-term, trait-like cognitive dysfunction.

The significant differences in PANSS ‘cognitive’ symptoms of insight and stereotyped thinking are of interest; the effects here were strong enough to be detected even in the small samples where test performances were doubly dissociated. Those who only failed the PAL showed less severe symptoms than those who failed both tests but are worse than those who failed only the IDED task. It is interesting that these symptoms were related to PAL failure and not to IDED failure, since poor insight and stereotyped thinking are typically thought of as symptoms of frontal dysfunction, and would therefore be expected to show associations with executive tasks.

The data set out herein demonstrates that deficits in visuospatial associative learning are dissociated from deficits in attention-shifting in patients early in their first episode of psychosis, regardless of the relative sensitivity of the two tests. Associative learning, but not attention-shifting performance, is associated with clinical and functional presentation. This provides indication that temporo-hippocampal dysfunction is more detrimental to well-being than fronto-striatal dysfunction in the early stages of psychosis, or alternately that executive function reflects more trait-like impairments and is less sensitive to symptom severity. The dissociation found here suggests no common pattern of cognitive impairment in psychosis, but rather that multiple factors including the patients premorbid strengths and weaknesses may be reflected in the impairments that show at onset of psychosis.

### REFERENCES

American Psychiatric Association (1994). DSM-IV: Diagnostic and Statistical Manual of Mental Disorders. (4th ed.). Washington, D.C.: American Psychiatric Association.
Beck A T, Steer R A (1987). Beck Depression Inventory. USA: The Psychological Corporation.
Bilder R M et al (2002) American Journal of Psychiatry 159, 1018-28.
Blackwell A D et al (2004). Dementia and Geriatric Cognitive Disorders 17, 42-8.
Blanchard J J, Neale J M (1994) American Journal of Psychiatry 151, 40-8.
Byrne M et al (1999) Psychological Medicine 29, 1161-73.
Cannon T D et al (1994) Archives of General Psychiatry 51, 651-61.
Chapman L J, Chapman J P (2001) Journal of Abnormal Psychology 110, 31-9.
Egan M F et al (2001) Biological Psychiatry 50, 98-107.
Elliott R et al (1995) Psychological Medicine 25, 619-30.
First M B et al (1997). Structured Clinical Interview for DSM-IV Axis I Disorders. Washington, D.C.: American Psychiatric Press.
Franke P et al, (1992) Schizophrenia Research 6, 243-9.
Goldberg T E et al (1988) International Journal of Neuroscience 42, 51-8.
Green M F et al (2000) Schizophrenia Bulletin 26, 119-136.
Heinrichs R W, Zakzanis K K (1998) Neuropsychology 12, 426-45.
Hutton S B et al (1998) Psychological Medicine 28, 463-73.
Joyce E et al (2002) British Journal of Psychiatry Supplement 43, s38-44.
Kay S R et al (1987) Schizophrenia Bulletin 13, 261-76.
Keefe R S et al (1999) Schizophrenia Bulletin 25, 201-22.
Kremen W S et al (1994) Schizophrenia Bulletin 20, 103-19.
Lawrence A D et al (1998) et al Brain 121 (Pt 7), 1329-41.
Lawrence A D et al (1996) Brain 119 (Pt 5), 1633-45.
Maguire E A et al (1998) Journal of Cognitive Neuroscience 10, 61-76.
McKenna P J et al (1990) Psychological Medicine 20, 967-72.
Miyashita Y et al (1998) Neurobiology of Learning and Memory 70, 197-211.
Mohamed S et al (1999). Archives of General Psychiatry 56, 749-54.
Nelson H (1982). The National Adult Reading Test. Windsor, UK: NFER Nelson.
Owen A M et al (1993a) Neuropsychologia 31, 627-44.
Owen A M et al (1996) Journal of Cognitive Neuroscience 8, 588-602.
Owen A M et al (1993b) Brain 116 (Pt 5), 1159-75.
Owen A M et al (1991) Neuropsychologia 29, 993-1006.
Pantelis C et al (2003a). Lancet 361, 281-8.
Pantelis C et al (2003b) Australian and New Zealand Journal of Psychiatry 37, 399-406.
Riley E M et al (2000) Schizophrenia Research 43, 47-55.
Robbins T W et al (1998) Journal of the International Neuropsychological Society 4, 474-490.
Roberts A C et al (1988) Quarterly Journal of Experimental Psychology Section B 40, 321-41.
Rogers R D et al (2000) Journal of Cognitive Neuroscience 12, 142-62.
Sahakian B J et al (1990) Neuropsychologia 28, 1197-213.
Sahakian B J et al (1988) Brain 111 (Pt 3), 695-718.
Sahakian B J et al (1992) Journal of the Royal Society of Medicine 85, 399-402.
Saykin A J et al (1994) Archives of General Psychiatry 51, 124-31.
Smith M L, Milner B (1981). Neuropsychologia 19, 781-93.
Swainson R et al (2001) Dementia and Geriatric Cognitive Disorders 12, 265-80.
Thompson P M et al (2001). Proceedings of the National Academy of Sciences of the United States of America 98, 11650-5.
Wolf L E et al (2002) Schizophrenia Research 57, 173.
Wood S J et al (2002). Psychological Medicine 32, 429-38.
Young R C et al (1978) British Journal of Psychiatry 133, 429-35.

TABLE 1

| | | | Association | Association |
|---|---|---|---|---|
| | | | with EDS | with PAL |
| | | | errors | errors |
| | Measure | n | (Rho) | (Rho) |
| Demographics | NART | 64 | −.15 | −.12 |
| | Age | 71 | .12 | .03 |
| | DUP this | 48 | −.03 | .16 |
| | episode | | | |
| | DUP lifetime | 59 | .00 | .07 |
| | history | | | |
| PANSS scale | Positive | 64 | −.05 | .22 |
| scores | Negative | 64 | .06 | .34** |
| | General | 64 | −.05 | .25 |
| Symptom | Young mania | 45 | −.13 | .32* |
| scales | Beck depression | 42 | .16 | −.39* |
| Global | Symptoms | 63 | .06 | −.22 |
| Assessment of | Disability | 64 | −.10 | −.41** |
| Function | Total | 64 | −.14 | −.28* |
| PANSS | Abstract | 62 | .26* | .29* |
| ‘cognitive’ | thinking | | | |
| symptoms | Flow of | 62 | −.04 | .14 |
| | conversation | | | |
| | Stereotyped | 62 | .00 | .39** |
| | thinking | | | |
| | Poor attention | 62 | .08 | .32* |
| | Insight | 62 | −.16 | .39** |

*p < 0.05
**p < 0.01

TABLE 2

| | | Mean (SD) for | | Mean (SD) for | | | | |
|---|---|---|---|---|---|---|---|---|
| | Measure | PAL passers | | PAL failers | | t | df | p |
| Demographics | NART | 110.4 | (7.68) | 109.3 | (9.54) | 0.467 | 62 | n.s |
| | Age | 28.3 | (10.82) | 24.6 | (7.14) | 1.562 | 69 | n.s |
| PANSS | PANSS General | 28.9 | (6.06) | 34.8 | (10.97) | −2.764 | 31.6 | <0.05 |
| scales | PANSS Positive | 15.2 | (5.85) | 17.8 | (5.64) | −1.783 | 62 | 0.08 |
| | PANSS Negative | 11.9 | (5.12) | 14.7 | (6.03) | −2.468 | 69 | <0.05 |
| Symptom | Young mania | 8.6 | (7.28) | 12.9 | (7.99) | −1.825 | 43 | 0.08 |
| scales | BDI | 20.7 | (12.34) | 19.2 | (17.20) | 0.328 | 40 | n.s |
| Global | GAF symptoms | 45.8 | (20.96) | 36.6 | (16.78) | 1.810 | 61 | 0.08 |
| Assessment | GAF disability | 58.5 | (18.64) | 45.3 | (19.57) | 2.697 | 62 | <0.01 |
| of Function | GAF total | 49.8 | (18.54) | 39.5 | (15.63) | 2.266 | 62 | <0.05 |
| | | | | | | z | n | p |
| PANSS | Abstract thinking | 1.97 | (1.16) | 2.36 | (1.18) | −1.630 | 68 | n.s |
| ‘cognitive’ | Flow of | 1.51 | (0.91) | 1.78 | (1.09) | −1.613 | 69 | n.s |
| symptoms | conversation | | | | | | | |
| | Stereotyped | 1.41 | (0.85) | 2.48 | (1.44) | −3.233 | 69 | <0.01 |
| | thinking | | | | | | | |
| | Poor attention | 1.38 | (0.85) | 1.87 | (1.10) | −2.039 | 69 | <0.05 |
| | Insight | 2.54 | (1.76) | 4.00 | (2.02) | −2.609 | 69 | <0.01 |

1. A method for assessing an individual having a first episode psychosis comprising;
determining the visuospatial associative learning ability of said individual,
wherein the visuospatial associative learning ability is indicative of the prognosis of the first episode psychosis in the individual.

2. A method according to claim 1 wherein a reduction in visuospatial associative learning relative to controls is indicative of the severity of the psychotic disorder and/or the probable clinical outcome in the individual.

3. A method according to claim 1 wherein visuospatial associative learning ability is determined using a paired associates learning test.

4. A method according to claim 3 wherein the paired associates learning test is non-verbal.

5. A method according to claim 4 wherein the paired associates learning test is a CANTAB PAL test.

6. A method according to claim 1 wherein the visuospatial associative learning ability of said individual is expressed as a visuospatial associative learning score.

7. A method according to claim 1 comprising determining the prognosis or severity of the first episode psychosis from the visuospatial associative learning score.

8. A method according to claim 7 wherein the prognosis is expressed as a grade which is indicative of probable clinical outcome.

9. A method according to claim 7 wherein the prognosis is expressed as a grade which is indicative of severity.

10. A method according to claim 1 comprising identifying the individual as having a severe form of psychotic disorder.

11. A method according to claim 1 comprising identifying the individual as having a psychotic disorder with a negative prognosis or probable clinical outcome.

12. A method of identifying a therapeutic agent useful in the treatment of a first episode psychosis comprising
administering a test compound to an individual having a first episode psychosis, and;
determining the visuospatial associative learning ability of said individual,
wherein an increase in visuospatial associative learning ability following said administration is indicative that the agent is effective in the treatment of the first episode psychosis.

13. A computer system comprising a display and a processor adapted for use in a method according to claim 1.

14. A computer system according to claim 13 comprising a graphic interface.

15. A computer system according to claim 14 wherein the graphic interface comprises a touch sensitive monitor

16. A computer program product carrying computer-readable code for performing the method of claim 1.

17. Computer-readable code for performing the method of claim 1.

18. A computer system configured to perform the method of claim 1.

19. A test device for assessing an individual with a first episode psychosis, comprising a display, a graphic interface and a processor adapted for use in method according to claim 1.

20. A method for determining the presence of psychosis in an individual having an at risk mental state (ARMS) comprising;
determining the visuospatial associative learning ability of said individual,
wherein the visuospatial associative learning ability is indicative of the presence or absence of psychosis.

21. A method according to claim 20 wherein a reduction in visuospatial associative learning relative to controls is indicative of the presence of psychosis.

22. A method according to claim 20 wherein visuospatial associative learning ability is determined using a paired associates learning test.

23. A method according to claim 22 wherein the paired associates learning test is non-verbal.

24. A method according to claim 23 wherein the paired associates learning test is a CANTAB PAL test.

25. A method according to claim 20 wherein the visuospatial associative learning ability of said individual is expressed as a visuospatial associative learning score.

26. A method according to claim 20 comprising determining the presence of psychosis from the visuospatial associative learning score.

27. A method according to claim 20 comprising identifying the individual as having a psychosis.

28. A computer system comprising a display and a processor adapted for use in a method according to claim 20.

29. A computer system according to claim 28 comprising a graphic interface.

30. A computer system according to claim 29 wherein the graphic interface comprises a touch sensitive monitor

31. A computer program product carrying computer-readable code for performing the method of claim 20.

32. Computer-readable code for performing the method of claim 20.

33. A computer system configured to perform the method of claim 20.

34. A test device for assessing an individual for psychosis, comprising a display, a graphic interface and a processor adapted for use in method according to claim 20.
